(19) United States
(12) Patent Application Publication
(10) Pub. No.: US 2022/0348975 A1
(43) Pub. Date: Nov. 3, 2022

(54) Apparatus and method for producing a bacterial cellulose composite having a core-shell structure by dynamic fermentation

(71) Applicants: Chunyan Zhong, Haikou City (CN); Yuguang Zhong, Haikou City (CN); Hainan Yeguo Foods Co., Ltd., Haikou City (CN); Hainan Guangyu Biotechnology Co., Ltd., Haikou City (CN); Nanjing Yeguo Foods Co., Ltd., Nanjing City (CN); Baoding Guangyu Fruit Processing Food Co., Ltd, Baoding City (CN)

(72) Inventors: Yuguang Zhong, Haikou City (CN); Chunyan Zhong, Haikou City (CN)

(21) Appl. No.: 17/858,403

(22) Filed: Jul. 6, 2022

Related U.S. Application Data
(63) Continuation of application No. PCT/CN2020/070900, filed on Jan. 8, 2020.

(51) Int. Cl. C12P 19/04; C12M 1/12; C12M 1/34; C08L 1/02

(52) U.S. Cl. CPC C12P 19/04; C12M 23/04; C12M 41/12; C08L 1/02; C08L 2207/53

(57) ABSTRACT

An apparatus and method for producing a bacterial cellulose composite having a core-shell structure by dynamic fermentation are described. The apparatus comprises a fermentation culture container and two rollers and two heating guide plates arranged in the fermentation culture container. The apparatus can realize dynamic fermentation and coating, and can obtain a bacterial cellulose composite material with controllable shape and size, good biocompatibility and safety.

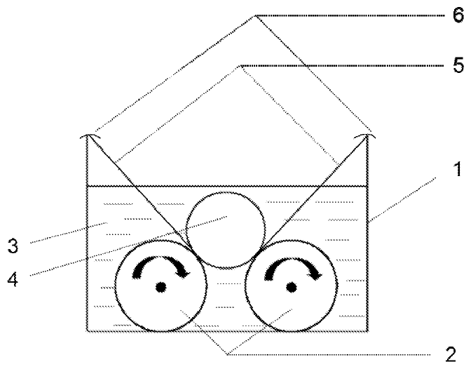

### CROSS-REFERENCE TO RELATED APPLICATIONS

This application is a continuation application of International Application No. PCT/CN2020/070900, filed Jan. 8, 2020, which is hereby incorporated by reference in its entirety.

### FIELD OF TECHNOLOGY

The invention belongs to the technical field of dynamic fermentation, and relates to an apparatus and method for producing a bacterial cellulose composite having a core-shell structure by dynamic fermentation.

### BACKGROUND ART

Cellulose is the most abundant biopolymer with great development potential on earth. It is not only a traditional raw material for the textile industry and paper industry, but also can be used to manufacture polymer composite materials and high-performance materials. It plays an important role in many high and new technology fields. Bacterial cellulose is a natural cellulose obtained by microbial fermentation, and is a polymer compound formed by connecting glucose with β-1,4-glycosidic chains. The bacterial cellulose has the same molecular building blocks as that of the natural celluloses produced by plants or algae, and it has many unique properties: (1) High crystallinity and degree of polymerization. As compared with plant cellulose, bacterial cellulose does not contain concomitant products such as lignin, pectin and hemicellulose, and has a high degree of crystallinity (up to 95%, compared to 65% for plant cellulose) and a high degree of polymerization (a DP value of 2000-8000). (2) Ultra-fine nano-network structure. Bacterial cellulose fibers are composed of microfibers with a diameter of 3-4 nanometers into fiber bundles with a thickness of 40-60 nanometers, and are intertwined to form a well-developed ultra-fine nano-network structure. (3) High elastic modulus and tensile strength. The elastic modulus of bacterial cellulose is several times to ten times that of common plant fibers, and its tensile strength is high. (4) High water retention values (WRV). The WRV value of undried bacterial cellulose is as high as 1000% or more, and the water retention value after freeze-drying is still more than 600%. The water retention value of bacterial cellulose after drying at 100° C. was comparable to that of cotton linters. (5) Excellent biocompatibility, adaptability and biodegradability. (6) Synthetic controllability. Due to these excellent properties, bacterial cellulose can be widely used in many fields, such as food, biomedicine, medical apparatuses, tissue engineering materials, etc.

Since bacterial cellulose has strong hydrophilicity, viscosity and stability, it can be used as food forming agent, thickener, dispersant, anti-solubilizer, as well as as casing and the skeleton of some foods to improve the taste. It has become a new important food base and dietary fiber. It has been widely used in foods such as jellies, milk tea, jams, desserts, etc., and is one of the most popular raw materials in commercial food. Due to its good biocompatibility, high mechanical strength in wet state, and good liquid and gas permeability, bacterial cellulose can be used as an excellent biological material in various fields of biomedicines. For example, Biofill and Gengiflex are two typical bacterial cellulose products that have been widely used as surgical and dental materials. Biofill has been successfully used as a temporary replacement for artificial skin having burns of second and third degree, ulcers, etc. Gengiflex has been used for the repair of periodontal membrane tissue. A novel biomaterial BASYC designed based on the in situ plasticity of bacterial cellulose is expected to be used as artificial blood vessels in microsurgery. At the same time, the reported applications also include the repair of bone, cornea, cartilage, tendon and other tissues.

The current fermentation and preparation technologies of bacterial cellulose are mainly divided into two categories: static fermentation and dynamic fermentation. The preparation of bacterial cellulose composites is mainly based on static fermentation. In the static fermentation process, various water-soluble polymers such as carboxymethyl cellulose, hemicellulose, chitosan, and gelatin are added to the culture solution to obtain different types of composite materials. Also, by using breathable materials as molds, different shapes of bacterial cellulose materials can be obtained during static fermentation. For example, in UK Patent GB 2,169,543, an oxygen-permeable hand-shaped mold is used to produce artificial skin in the shape of a glove; in EP Patent 0,396,344 and JP Patent 3,272,772, an artificial blood vessel was prepared by injecting the culture solution containing bacteria by using an oxygen-permeable hollow tube under static fermentation conditions. In terms of dynamic fermentation, conventional paddle stirring can only produce granular bacterial celluloses. The Rotating disc fermentor designed by Krystynowicz A et al. solved the problem that bacterial cellulose is difficult to form a film during dynamic culture, and a disc-shaped bacterial cellulose film is produced (Journal of Industrial Microbiology & Biotechnology 2002(29):189-195). Based on this, Chinese patent CN2937138Y discloses a rotary disk type fermentation reaction apparatus, but this type of apparatus can only obtain disc-shaped bacterial cellulose membranes. In order to further obtain bacterial cellulose materials with controllable shape, Chinese patent CN101914434A designs a method for dynamically preparing special-shaped cavity bacterial cellulose materials, by which a cavity bacterial cellulose material with a certain cross-sectional shape can be obtained. However, in this method, the mold must pass through the rotating shaft, and thus a completely airtight bacterial cellulose coating cannot be obtained.

In conclusion, the design of a reasonable, simple, and feasible bacterial cellulose fermentation and culture apparatus and a method for producing the desired composite material with a certain morphology and complete coating have great practical significance and commercialization prospects.

### SUMMARY OF THE INVENTION

An object of the present invention is to provide an apparatus for producing a bacterial cellulose composite having a core-shell structure by dynamic fermentation. Another object of the present invention is to provide a method for producing a bacterial cellulose composite having a core-shell structure by dynamic fermentation. Another object of the present invention is to provide the bacterial cellulose composite having a core-shell structure produced by the method.

The objects of the present invention are achieved through the following technical solutions.

In an aspect, the present invention provides an apparatus for producing a bacterial cellulose composite having a core-shell structure by dynamic fermentation, comprising:

a fermentation culture container and two rollers and two heating guide plates arranged in the fermentation culture container;

both ends of the rotating shafts of the two rollers are respectively movably connected to the inner wall of the fermentation culture container; the two rollers are arranged in parallel in the horizontal direction; there is a gap between the rollers, and the distance between the rotating shafts of the rollers is adjustable;

the two heating guide plates are parallel to the rotating shafts of the two rollers, wherein one end of one of the heating guide plates is movably connected to the fermentation culture container (preferably via a fastener), and the other end extends obliquely downward to be above the gap between the two rollers and abuts against one of the rollers; one end of the other heating guide plate is movably connected to the fermentation culture container via the fastener, and the other end extends obliquely downward to be above the gap between the two rollers and abuts against the other roller.

The apparatus provided by the invention can realize dynamic fermentation and coating, and can obtain a bacterial cellulose composite material with controllable shape and size by coating. This apparatus is simple, easy to operate and has high yield, and is suitable for industrial production. The obtained coated bacterial cellulose composite material can be widely used in the fields of biomedicine and medical apparatuses, such as bacterial cellulose coating on the surface of implant medical apparatuses, sustained-release drugs or micro apparatuses, or can also be used as a coating for food or food materials, and the like.

In the apparatus as described above, preferably, the apparatus further comprises a driving member for driving the two rollers to rotate; more preferably, the driving member is used for driving the two rollers to rotate in the same direction (clockwise or counter-clockwise).

In the apparatus as described above, preferably, the apparatus further comprises a heating member for heating the two heating guide plates. The heating member may be an external heating apparatus that makes the heating guide plate generate heat, or a heating member arranged in the heating guide plate itself.

In the apparatus as described above, preferably, the two rollers are cylindrical rollers of the same shape and size.

In the apparatus as described above, preferably, each of the two heating guide plates has an angle with the inner side wall of the fermentation culture container of 15-60 degrees. The two heating guide plates are movable and removable articulating fittings. Fasteners can be used to fix them on be above the fermentation culture container, and the inclination angle of the two heating guide plates can be adjusted by the fasteners to adjust the heated liquid flow rate of the thermoplastic polymer.

In another aspect, the present invention further provides a method for producing a bacterial cellulose composite having a core-shell structure by performing dynamic fermentation using the apparatus as described above. The method comprises the following steps:

adjusting the distance between of the rollers so that the minimum width of the gap is smaller than the diameter or size of the core material (the size is such that the core material can be clamped above the two rolls); sterilizing the core material to be coated, and then placing it above the gap between the two rollers to ensure that the core material can abut against each of the two rollers above the gap; setting the rotation speed and rotation direction of the rollers, so that the core material can realize vibration (including rotation) without horizontal displacement according to the rotation of the rollers;

setting the length of the heating guide plates and adjusting the angle of the heating guide plates to ensure that one end of the two heating guide plates is at the gap where the core material abuts against the rollers (this can not only ensure that the rollers rotate with the core material, but also make the thermoplastic polymer flow down the heating guide plates and coat the surface of the core material through the rotation of the rollers);

formulating a bacterial cellulose fermentation culture solution and sterilizing it at high pressure, and then mixing it with strain seed mash to obtain a fermentation mixed solution;

pouring the fermentation mixed solution into the fermentation culture container to immerse the core material, and starting the rollers to rotate in the same direction for dynamic fermentation; after the completion of fermentation, discharging fermentation broth, at which the outer layer of the core material is coated with bacterial cellulose obtained by strain fermentation to form a core material-bacterial cellulose composite; starting the heating guide plates and allowing the heated liquid of thermoplastic polymeric polyethylene to flow down along the heating guide plates at both sides, respectively; with the rotation of the rollers, uniformly coating the surface of the core material-bacterial cellulose composite with the thermoplastic polymer, thereby producing a bacterial cellulose composite having a core-shell structure; alternatively,

starting the heating guide plates and allowing the heated liquid of thermoplastic polymeric polyethylene to flow down along the heating guide plates at both sides, respectively; with the rotation of the rollers, uniformly coating the surface of the core material with the thermoplastic polymer, thereby producing a core material-thermal plastic polymer composite; pouring the fermentation mixed solution into the fermentation culture container to immerse the rollers, and starting the rollers to rotate in the same direction for dynamic fermentation; after the completion of fermentation, discharging fermentation broth, at which the outer layer of core material-thermal plastic polymer composite is coated with bacterial cellulose obtained by strain fermentation, thereby producing a bacterial cellulose composite having a core-shell structure.

In the present invention, the coating amount of bacterial cellulose and the coating amount of thermoplastic polymer material can be reasonably set according to actual needs.

In the dynamic fermentation process of the present invention, the structure of the obtained bacterial cellulose composite having a core-shell structure from inside to outside can be: “core material-bacterial cellulose-thermal plastic polymer”, “core material-thermal plastic polymer-bacterial cellulose”, or the like.

In the method as described above, preferably, the method also includes repeating the step of coating bacterial cellulose by dynamic fermentation and/or the step of coating thermoplastic polymer to obtain a bacterial cellulose composite having a core-shell structure with more layers. Taking such repeated steps can obtain bacterial cellulose composites with different layered structures, for example, the one with a structure from inside to outside of “core material-bacterial cellulose-thermal plastic polymer-bacterial cellulose-thermal plastic polymer”, “core material-thermal plastic polymer-bacterial cellulose-thermal plastic polymer-bacterial cellulose”, or the like.

In the method as described above, preferably, the core material comprises one or a combination of more of inorganic materials, organic polymer materials and metal materials.

In the method as described above, preferably, the shape of the core material is spherical, quasi-spherical, cylindrical, rod-shaped or any irregular body.

In the method as described above, preferably, the thermoplastic polymer material comprises one or a combination of more of polyethylene, polypropylene, polystyrene, polymethyl methacrylate, nylon, polyurethane, polyester and polylactic acid.

In the method as described above, preferably, the heating temperature of the heating guide plates is 50-300° C.

In the method as described above, preferably, the strains comprise one or a combination of more of Acetobacter xylinum, Rhizobium, Sporosarcina, Pseudomonas, Achromobacter, Alcaligenes, Aerobacter, and Azotobacter.

In the method as described above, preferably, the added amount of strain seed mash is 1-5 wt % the fermentation culture solution.

In the method as described above, preferably, the dynamic fermentation is performed at a temperature of 20-30° C. for 3-30 days.

In the method as described above, preferably, when the dynamic fermentation is performed, the rotation speeds of the two rollers are the same, which are both 0.1-60 rpm; preferably 4-20 rpm.

During the dynamic culture of bacterial cellulose, the flow rate of the culture solution must be controlled within a certain range. If the flow rate is too fast, the bacterial species is easy to mutate, and it is difficult for the bacterial cellulose to adhere to the core material, so that only granular bacterial cellulose can be obtained in the culture solution. When the flow rate is slow, bacterial cellulose will be produced in large quantities on the gas-liquid interface of the culture container, which affects the coating of bacterial cellulose on the core material. In addition, the flow rate of the culture solution also affects the yield of bacterial cellulose, the flatness of the coating on the surface of the core material, and the three-dimensional network structure of the nanofibers that constitute the bacterial cellulose. A preferred condition is that the rotation rate of the rollers is controlled within the range of 4-20 rpm.

In the method as described above, preferably, during the process of dynamic fermentation, adding 0.1-5 wt% soluble additives to the fermentation mixed solution is further included. The soluble additives comprise one or a combination of more of gelatin, sodium hyaluronate, starch, pectin, chitosan, sodium alginate, and soluble cellulose derivatives.

The applicant's research found that adding soluble macromolecules in situ during the bacterial cellulose culture process can change the physical and chemical properties of the bacterial cellulose, producing a composite material that meet various applications. The dynamic fermentation method adopted in the present invention can make the bacterial cellulose complex with the soluble macromolecule during the fermentation and culture process while coating the core material, thereby expanding the application scope of the bacterial cellulose composite material.

In the method as described above, preferably, after the dynamic fermentation is completed, a purification step of the bacterial cellulose-coated composite is also included. The purification step is performed as follows: the bacterial cellulose-coated composite is washed in an aqueous NaOH solution with a mass percentage of 4% to 8% at a temperature of 70-100° C. for 4-6 h, and then repeatedly rinsed with distilled water until neutral. By purification, the bacterial proteins on the bacterial cellulose and the residual medium adhering to the cellulose membrane are removed, such that the bacterial cellulose-coated composite can reach the pharmaceutical or food grade.

In yet another aspect, the present invention provides a bacterial cellulose composite having a core-shell structure produced by the above method, of which the inner core layer is a core material, and the core material is coated with a single layer or multiple layers of bacterial cellulose and/or thermoplastic polymer materials.

The present invention has the following beneficial effects:

(1) The apparatus provided by the invention can realize dynamic fermentation and coating, and can obtain a bacterial cellulose composite material with controllable shape and size by coating. This apparatus is simple, easy to operate and has high yield, and is suitable for industrial production. The obtained coated bacterial cellulose composite material can be widely used in the fields of biomedicine and medical apparatuses, such as bacterial cellulose coating on the surface of implant medical apparatuses, sustained-release drugs or micro apparatuses, or can also be used as a coating for food or food materials, and the like.

(2) The dynamic fermentation method of the present invention can be performed without using any toxic solvent. This will not cause problems such as environmental pollution and ecological hazards, and meets the requirements for medical or edible use. Adding various water-soluble polymers in the fermentation process can obtain bacterial cellulose composite materials with different properties, and bring excellent biocompatibility and biosafety.

(3) Using the dynamic fermentation method of the present invention, a bacterial cellulose composite having a core-shell structure layered differently can be rapidly obtained, which can be widely used in many fields such as biomedicine, medical equipment, and food.

### DESCRIPTION OF THE FIGURE

The FIGURE is a schematic structural diagram of an apparatus for producing a bacterial cellulose composite having a core-shell structure by dynamic fermentation in an embodiment of the present invention.

### REFERENCE NUMBERS

1. fermentation culture container,
2. rollers,
3. fermentation culture solution,
4. core material to be coated,
5. heating guide plates,
6. fasteners.

### DETAILED DESCRIPTION OF PREFERRED EMBODIMENTS

In order to have a clearer understanding of the technical features, purposes and beneficial effects of the present invention, the technical solutions of the present invention are now described in detail below, but should not be construed as limiting the scope of implementation of the present invention.

The experimental methods used in the following examples are conventional methods, unless otherwise specified.

The materials, reagents, etc. used in the following examples can be obtained from commercial sources, unless otherwise specified.

### EXAMPLE 1

This example provides an apparatus for producing a bacterial cellulose composite having a core-shell structure by dynamic fermentation. As shown in the FIGURE, the apparatus comprises:

a fermentation culture container 1 and two rollers 2 and two heating guide plates 5 arranged in the fermentation culture container. Both ends of the rotating shafts of the two rollers 2 are respectively movably connected to the inner wall of the fermentation culture container; the two rollers are arranged in parallel in the horizontal direction; there is a gap between the rollers, and the distance between the rotating shafts of the rollers is adjustable; the two heating guide plates 5 are parallel to the rotating shafts of the two rollers 2, wherein one end of one of the heating guide plates 5 is movably connected to the fermentation culture container 1 via a fastener 6, and the other end extends obliquely downward to be above the gap between the two rollers and abuts against one of the rollers; one end of the other heating guide plate is movably connected to the fermentation culture container via the fastener 6, and the other end extends obliquely downward to be above the gap between the two rollers and abuts against the other roller 2. The two rollers are preferably cylindrical rollers of the same shape and size.

During dynamic fermentation, the core material to be coated 4 is located above the gap between the two rollers 2 and abuts against the rollers; and the fermentation culture solution 3 is loaded in the fermentation culture container 1. The apparatus further comprises a driving member for driving the two rollers to rotate in the same direction. The apparatus further comprises a heating member for heating the two heating guide plates.

In another aspect, the example also provides a method for producing a bacterial cellulose composite having a core-shell structure by performing dynamic fermentation using the apparatus in the example as described above. The method comprises the following steps:

(1) Two cylindrical glass rollers of the same shape and size that can rotate clockwise in the same direction at a constant speed were arranged in parallel in a fermentation culture container with an upward opening. A spherical polyurethane material (core material) with a diameter of 30 mm was placed between the two rollers, as shown in the FIGURE, the distance between the two rollers was adjusted to 26 mm, and the rotation speed was 4 rpm. The spherical polyurethane can realize vibration without horizontal displacement according to the rotation of the rollers.

(2) Two heating guide plates were fixed on top of the fermentation culture container, and each of the two heating guide plates is adjusted to have an angle with the side of the fermentation culture container of 30 degrees, to ensure that one end of the two heating plates is located at the gap where the spherical polyurethane abuts against the roller.

(3) Acetobacter xylinum that can secrete bacterial cellulose was activated to prepare a seed mash, and the seed mash with a strain concentration of 1 wt % and a fermentation medium were then mixed to obtain a fermentation mixed solution; wherein, the fermentation medium was a high-temperature sterilized medium, and the components of the medium were the commonly used components for bacterial cellulose fermentation in this field.

(4) The fermentation mixed solution was poured into the fermentation culture container to immerse the core material. The rollers were started to rotate in the same direction for dynamic fermentation, and the fermentation was carried out at 35° C. for 3 days. After the fermentation was completed, the fermentation broth was discharged. At this time, the outer layer of spherical polyurethane was coated with bacterial cellulose obtained by strain fermentation. The product was immersed an aqueous NaOH solution with a mass percentage of 4% and heated at 100° C. for 6 h, and then repeatedly rinsed with distilled water until neutral, to form a spherical polyurethane-bacterial cellulose composite.

(5) The heating guide plates were started, and the heating temperature was adjusted to 190° C. The heated liquid of thermoplastic polymeric polyethylene flowed down along the heating guide plates at both sides, respectively. With the rotation of the rollers, the thermoplastic polymer was uniformly coated on the surface of the core material-bacterial cellulose composite, thereby producing a core-shell structural composite material in which the surface of the spherical polyurethane material was uniformly covered with polyethylene/bacterial cellulose.

### EXAMPLE 2

This example provides a method for producing a bacterial cellulose composite having a core-shell structure by performing dynamic fermentation using the apparatus in the Example 1 as described above. The method comprises the following steps:

(1) Two cylindrical glass rollers of the same shape and size that can rotate clockwise in the same direction at a constant speed were arranged in parallel in a fermentation culture container with an upward opening. A bioceramic (core material) with a diameter of 50 mm and a length of 70 mm was placed between the two rollers, as shown in the FIGURE, the distance between the two rollers was adjusted to 40 mm, and the rotation speed was 8 rpm. The bioceramic can realize vibration without horizontal displacement according to the rotation of the rollers.

(2) Two heating guide plates were fixed on top of the fermentation culture container, and each of the two heating guide plates is adjusted to have an angle with the side of the fermentation culture container of 45 degrees, to ensure that one end of the two heating plates is located at the gap where the bioceramic abuts against the roller.

(3) Rhizobium that can secrete bacterial cellulose was activated to prepare a seed mash, and the seed mash with a strain concentration of 2 wt % and a fermentation medium were then mixed to obtain a fermentation mixed solution; wherein, the fermentation medium was a high-temperature sterilized medium, the components of the medium were the commonly used components for bacterial cellulose fermentation in this field, and the fermentation mixed solution further contained 0.1 wt % gelatin.

(4) The fermentation mixed solution was poured into the fermentation culture container to immerse the core material. The rollers were started to rotate in the same direction for dynamic fermentation, and the fermentation was carried out at 20° C. for 30 days. After the fermentation was completed, the fermentation broth was discharged. At this time, the outer layer of spherical polyurethane was coated with bacterial cellulose obtained by strain fermentation. The product was immersed an aqueous NaOH solution with a mass percentage of 5% and heated at 90° C. for 5 h, and then repeatedly rinsed with distilled water until neutral, to form a bioceramic-bacterial cellulose composite.

(5) The heating guide plates were started, and the heating temperature was adjusted to 230° C. The heated liquid of thermoplastic polymeric polypropylene flowed down along the heating guide plates at both sides, respectively. With the rotation of the rollers, the thermoplastic polymer was uniformly coated on the surface of the bioceramic-bacterial cellulose composite, thereby producing a core-shell structural composite material in which the surface of the bioceramic was uniformly covered with polypropylene/bacterial cellulose.

### EXAMPLE 3

This example provides a method for producing a bacterial cellulose composite having a core-shell structure by performing dynamic fermentation using the apparatus in the Example 1 as described above. The method comprises the following steps:

(1) Two cylindrical stainless steel rollers of the same shape and size that can rotate clockwise in the same direction at a constant speed were arranged in parallel in a fermentation culture container with an upward opening. A titanium alloy orthopedic implant screw of 6.5 mm (core material) was placed between the two rollers, as shown in the FIGURE, the distance between the two rollers was adjusted to 5 mm, and the rotation speed was 12 rpm. The screw can realize vibration without horizontal displacement according to the rotation of the rollers.

(2) Two heating guide plates were fixed on top of the fermentation culture container, and each of the two heating guide plates is adjusted to have an angle with the side of the fermentation culture container of 45 degrees, to ensure that one end of the two heating plates is located at the gap where the screw abuts against the roller.

(3) Sporosarcina that can secrete bacterial cellulose was activated to prepare a seed mash, and the seed mash with a strain concentration of 3 wt % and a fermentation medium were then mixed to obtain a fermentation mixed solution; wherein, the fermentation medium was a high-temperature sterilized medium, the components of the medium were the commonly used components for bacterial cellulose fermentation in this field, and the fermentation mixed solution further contained 1 wt % sodium hyaluronate and sodium alginate at a mass ratio of 1:1.

(4) The fermentation mixed solution was poured into the fermentation culture container to immerse the core material. The rollers were started to rotate in the same direction for dynamic fermentation, and the fermentation was carried out at 25° C. for 5 days. After the fermentation was completed, the fermentation broth was discharged. At this time, the outer layer of the screw was coated with bacterial cellulose obtained by strain fermentation. The product was immersed an aqueous NaOH solution with a mass percentage of 6% and heated at 80° C. for 4 h, and then repeatedly rinsed with distilled water until neutral, to form a screw-bacterial cellulose composite.

(5) The heating guide plates were started, and the heating temperature was adjusted to 200° C. The heated liquid of thermoplastic ethylene propylene copolymer flowed down along the heating guide plates at both sides, respectively. With the rotation of the rollers, the thermoplastic polymer was uniformly coated on the surface of the screw-bacterial cellulose composite, thereby producing a core-shell structural composite material in which the surface of the screw was uniformly covered with ethylene propylene copolymer/bacterial cellulose.

### EXAMPLE 4

This example provides a method for producing a bacterial cellulose composite having a core-shell structure by performing dynamic fermentation using the apparatus in the Example 1 as described above. The method comprises the following steps:

(1) Two cylindrical polytetrafluoroethylene rollers of the same shape and size that can rotate clockwise in the same direction at a constant speed were arranged in parallel in a fermentation culture container with an upward opening. A rod-like material of nano-hydroxyapatite-filled polymethyl methacrylate with a diameter of 2 mm (core material) was placed between the two rollers, as shown in the FIGURE, the distance between the two rollers was adjusted to 1.5 mm, and the rotation speed was 2 rpm. The rod material can realize vibration without horizontal displacement according to the rotation of the rollers.

(2) Two heating guide plates were fixed on top of the fermentation culture container, and each of the two heating guide plates is adjusted to have an angle with the side of the fermentation culture container of 60 degrees, to ensure that one end of the two heating plates is located at the gap where the rod material abuts against the roller.

(3) Pseudomonas that can secrete bacterial cellulose was activated to prepare a seed mash, and the seed mash with a strain concentration of 4 wt % and a fermentation medium were then mixed to obtain a fermentation mixed solution; wherein, the fermentation medium was a high-temperature sterilized medium, the components of the medium were the commonly used components for bacterial cellulose fermentation in this field, and the fermentation mixed solution further contained 2 wt % soluble starch.

(4) The fermentation mixed solution was poured into the fermentation culture container to immerse the core material. The rollers were started to rotate in the same direction for dynamic fermentation, and the fermentation was carried out at 30° C. for 7 days. After the fermentation was completed, the fermentation broth was discharged. At this time, the outer layer of rod material was coated with bacterial cellulose obtained by strain fermentation. The product was immersed an aqueous NaOH solution with a mass percentage of 7% and heated at 70° C. for 6 h, and then repeatedly rinsed with distilled water until neutral, to form a rod material-bacterial cellulose composite.

(5) The heating guide plates were started, and the heating temperature was adjusted to 270° C. The heated liquid of thermoplastic polymer nylon flowed down along the heating guide plates at both sides, respectively. With the rotation of the rollers, the thermoplastic polymer was uniformly coated on the surface of the rod material-bacterial cellulose composite, thereby producing a core-shell structural composite material in which the surface of the rod material was uniformly covered with nylon/bacterial cellulose.

### EXAMPLE 5

This example provides a method for producing a bacterial cellulose composite having a core-shell structure by performing dynamic fermentation using the apparatus in the Example 1 as described above. The method comprises the following steps:

(1) Two cylindrical plastic rollers of the same shape and size that can rotate clockwise in the same direction at a constant speed were arranged in parallel in a fermentation culture container with an upward opening. An irregular silicone rubber (core material) for filling breasts was placed between the two rollers, as shown in the FIGURE, the distance between the two rollers was adjusted to 70 mm, and the rotation speed was 30 rpm. The silicone rubber can realize vibration without horizontal displacement according to the rotation of the rollers.

(2) Two heating guide plates were fixed on top of the fermentation culture container, and each of the two heating guide plates is adjusted to have an angle with the side of the fermentation culture container of 45 degrees, to ensure that one end of the two heating plates is located at the gap where the silicone rubber abuts against the roller.

(3) Achromobacter and Alcaligenes that can secrete bacterial cellulose was activated to prepare a seed mash, and the seed mash with a strain concentration of 5 wt % and a fermentation medium were then mixed to obtain a fermentation mixed solution; wherein, the fermentation medium was a high-temperature sterilized medium, the components of the medium were the commonly used components for bacterial cellulose fermentation in this field, and the fermentation mixed solution further contained 3 wt % pectin.

(4) The fermentation mixed solution was poured into the fermentation culture container to immerse the core material. The rollers were started to rotate in the same direction for dynamic fermentation, and the fermentation was carried out at 31° C. for 15 days. After the fermentation was completed, the fermentation broth was discharged. At this time, the outer layer of silicone rubber was coated with bacterial cellulose obtained by strain fermentation. The product was immersed an aqueous NaOH solution with a mass percentage of 8% and heated at 70° C. for 5 h, and then repeatedly rinsed with distilled water until neutral, to form a silicone rubber-bacterial cellulose composite.

(5) The heating guide plates were started, and the heating temperature was adjusted to 190° C. The heated liquid of thermoplastic polymeric polyethylene flowed down along the heating guide plates at both sides, respectively. With the rotation of the rollers, the thermoplastic polymer was uniformly coated on the surface of the silicone rubber-bacterial cellulose composite, thereby producing a core-shell structural composite material in which the surface of the silicone rubber was uniformly covered with polyethylene/bacterial cellulose.

### EXAMPLE 6

This example provides a method for producing a bacterial cellulose composite having a core-shell structure by performing dynamic fermentation using the apparatus in the Example 1 as described above. The method comprises the following steps:

(1) Two cylindrical plastic rollers of the same shape and size that can rotate clockwise in the same direction at a constant speed were arranged in parallel in a fermentation culture container with an upward opening. A cobalt-chromium-molybdenum alloy artificial hip joint of irregular shape (core material) was placed between the two rollers, as shown in the FIGURE, the distance between the two rollers was adjusted to 50 mm, and the rotation speed was 15 rpm. The alloy can realize vibration without horizontal displacement according to the rotation of the rollers.

(2) Two heating guide plates were fixed on top of the fermentation culture container, and each of the two heating guide plates is adjusted to have an angle with the side of the fermentation culture container of 60 degrees, to ensure that one end of the two heating plates is located at the gap where the alloy abuts against the roller.

(3) Aerobacter and Azotobacter that can secrete bacterial cellulose was activated to prepare a seed mash, and the seed mash with a strain concentration of 3 wt % and a fermentation medium were then mixed to obtain a fermentation mixed solution; wherein, the fermentation medium was a high-temperature sterilized medium, the components of the medium were the commonly used components for bacterial cellulose fermentation in this field, and the fermentation mixed solution further contained 5 wt % chitosan and sodium carboxymethyl cellulose at a mass ratio of 1:3.

(4) The fermentation mixed solution was poured into the fermentation culture container to immerse the core material. The rollers were started to rotate in the same direction for dynamic fermentation, and the fermentation was carried out at 32° C. for 10 days. After the fermentation was completed, the fermentation broth was discharged. At this time, the outer layer of the alloy was coated with bacterial cellulose obtained by strain fermentation. The product was immersed an aqueous NaOH solution with a mass percentage of 6% and heated at 90° C. for 4 h, and then repeatedly rinsed with distilled water until neutral, to form an alloy-bacterial cellulose composite.

(5) The heating guide plates were started, and the heating temperature was adjusted to 190° C. The heated liquid of thermoplastic polymeric polyethylene flowed down along the heating guide plates at both sides, respectively. With the rotation of the rollers, the thermoplastic polymer was uniformly coated on the surface of the alloy-bacterial cellulose composite, thereby producing a core-shell structural composite material in which the surface of the alloy was uniformly covered with polyethylene/bacterial cellulose.

### Performance Test Experiments:

The following performance tests were performed on the core-shell structural composite material in which the surface of the spherical polyurethane material was uniformly covered with polyethylene/bacterial cellulose prepared in the Example 1.

Biocompatibility test: in accordance with GB/T 16886 Biological evaluation of medical apparatuses, the composite material (Example 1) and the polyurethane material (the core material of Example 1) evaluated for cytotoxicity, delayed contact sensitization in guinea pigs, skin irritation, etc.

The results show that the polyurethane material (the core material of Example 1) had a cytotoxicity of grade 2 and skin sensitization response; the composite material (Example 1) had a cytotoxicity of less than grade 2, no skin sensitization response and no intradermal irritation response, and have good biological safety. This indicated that the use of this patent improves the biocompatibility of the material.

1. An apparatus for producing a bacterial cellulose composite having a core-shell structure through a dynamic fermentation, comprising:
a fermentation culture container, and two rollers and two heating guide plates arranged in the fermentation culture container;
both ends of the rotating shafts of the two rollers are respectively movably connected to the inner wall of the fermentation culture container; the two rollers are arranged in parallel in the horizontal direction with a gap therebetween; and the distance between the rotating shafts of the rollers is adjustable;
the two heating guide plates are parallel to the rotating shafts of the two rollers, wherein one end of one of the heating guide plates is movably connected to the fermentation culture container, and the other end thereof extends obliquely downward to be above the gap between the two rollers and abuts against one of the rollers; and one end of the other heating guide plate is movably connected to the fermentation culture container, and the other end thereof extends obliquely downward to be above the gap between the two rollers and abuts against the other roller.

2. The apparatus according to claim 1, wherein the apparatus further comprises a driving member for driving the two rollers to rotate.

3. The apparatus according to claim 1, wherein the driving member is used for driving the two rollers to rotate in the same direction.

4. The apparatus according to claim 1, wherein the apparatus further comprises a heating member for heating the two heating guide plates.

5. The apparatus according to claim 1, wherein the two rollers are cylindrical rollers of the same shape and size.

6. The apparatus according to claim 1, wherein each of the two heating guide plates has an angle with the inner side wall of the fermentation culture container of 15-60 degrees.

7. A method for producing a bacterial cellulose composite having a core-shell structure by performing dynamic fermentation using the apparatus according to claim 1, comprising:
adjusting the distance between the rollers so that the minimum width of the gap is smaller than the diameter or length of the core material; sterilizing the core material to be coated, and then placing it above the gap between the two rollers to ensure that the core material can abut against each of the two rollers above the gap; setting the rotation speed and rotation direction of the rollers, so that the core material can realize vibration without horizontal displacement according to the rotation of the rollers;
setting the length of the heating guide plates and adjusting the angle of the heating guide plates to ensure that one end of the two heating guide plates is at the gap where the core material abuts against the rollers;
formulating a bacterial cellulose fermentation culture solution and sterilizing it at high pressure, and then mixing it with strain seed mash to obtain a fermentation mixed solution;
pouring the fermentation mixed solution into the fermentation culture container to immerse the core material, and starting the rollers to rotate in the same direction for dynamic fermentation; after the completion of fermentation, discharging fermentation broth, at which the outer layer of the core material is coated with bacterial cellulose obtained by strain fermentation to form a core material-bacterial cellulose composite; starting the heating guide plates and allowing the heated liquid of thermoplastic polymer to flow down along the heating guide plates at both sides, respectively; with the rotation of the rollers, uniformly coating the surface of the core material-bacterial cellulose composite with the thermoplastic polymer, thereby producing a bacterial cellulose composite having a core-shell structure; alternatively,
starting the heating guide plates and allowing the heated liquid of thermoplastic polymer to flow down along the heating guide plates at both sides, respectively; with the rotation of the rollers, uniformly coating the surface of the core material with the thermoplastic polymer, thereby producing a core material-thermal plastic polymer composite; pouring the fermentation mixed solution into the fermentation culture container to immerse the rollers, and starting the rollers to rotate in the same direction for dynamic fermentation; after the completion of fermentation, discharging fermentation broth, at which the outer layer of core material-thermal plastic polymer composite is coated with bacterial cellulose obtained by strain fermentation, thereby producing a bacterial cellulose composite having a core-shell structure.

8. The method according to claim 7, wherein the method also includes repeating coating bacterial cellulose by dynamic fermentation and/or coating thermoplastic polymer to obtain a bacterial cellulose composite having a core-shell structure with more layers.

9. The method according to claim 7, wherein the core material comprises one or a combination of more of inorganic materials, organic polymer materials and metal materials.

10. The method according to claim 7, wherein the shape of the core material is spherical, quasi-spherical, cylindrical, rod-shaped or any irregular body.

11. The method according to claim 7, wherein the thermoplastic polymer comprises one or a combination of more of polyethylene, polypropylene, polystyrene, polymethyl methacrylate, nylon, polyurethane, polyester and polylactic acid.

12. The method according to claim 7, wherein the heating temperature of the heating guide plates is 50-300° C.

13. The method according to claim 7, wherein the strains comprise one or a combination of more of Acetobacter xylinum, Rhizobium, Sporosarcina, Pseudomonas, Achromobacter, Alcaligenes, Aerobacter, and Azotobacter.

14. The method according to claim 7, wherein the added amount of strain seed mash is 1-5 wt % of the fermentation culture solution.

15. The method according to claim 7, wherein the dynamic fermentation is performed at a temperature of 20-30° C. for 3-30 days.

16. The method according to claim 7, wherein when the dynamic fermentation is performed, the rotation speeds of the two rollers are the same, which are both 0.1-60 rpm.

17. The method according to claim 7, wherein when the dynamic fermentation is performed, the rotation speeds of the two rollers are the same, which are both 4-20 rpm.

18. The method according to claim 7, wherein, during the process of dynamic fermentation, adding 0.1-5 wt % soluble additives to the fermentation mixed solution is further included; and the soluble additives comprise one or a combination of more of gelatin, sodium hyaluronate, starch, pectin, chitosan, sodium alginate, and soluble cellulose derivatives.

19. The method according to claim 7, wherein, after the dynamic fermentation is completed, further comprising purifying the bacterial cellulose-coated composite by washing the bacterial cellulose-coated composite in an aqueous NaOH solution with a mass percentage of 4% to 8% at a temperature of 70-100° C. for 4-6 h, and then repeatedly rinsing with distilled water until neutral.

20. A bacterial cellulose composite having a core-shell structure produced by the method according to claim 7, of which the inner core layer is a core material, which is coated with a single layer or multiple layers of bacterial cellulose and/or thermoplastic polymer.
